(19) United States
(12) Patent Application Publication
(10) Pub. No.: US 2007/0020673 A1
(43) Pub. Date: Jan. 25, 2007

(54) Model of autoimmune disease and methods for identifying agents against autoimmune disease

(76) Inventors: Patrick Daniel Matthias, Basel (CH); Jian Sun, Shanghai (CN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/501,626

(22) Filed: Aug. 8, 2006

Related U.S. Application Data
(63) Continuation of application No. 10/487,116, filed on Apr. 6, 2004, now abandoned.

(30) Foreign Application Priority Data
Aug. 22, 2001 (GB) 0120441.1

(51) Int. Cl. C12Q 1/68; C12N 5/06; C12N 5/08; A61K 48/00

(52) U.S. Cl. 435/6; 514/44; 435/366; 435/354

(57) ABSTRACT

Homozygouse knock-out mice lacking the Aiolos gene are shown to exhibit multiple phenotypes in common with humans suffering from the autoimmune disease Systemic Lupus Erythematosus (SLE). When Aiolos −/− mice are crossed with homozygous knock out mice lacking the OBF-1 transcription factor gene, resultant double knock out mice lack all signs of SLE. Methods of screening for agents active against autoimmune diseases, for example SLE are provided. In vitro methods include screening for antagonists of OBF-1, screening for agents which inhibit binding of OBF-1 to oct-1 or oct-2, screening for agonists or antagonists of Aiolos protein and screening for agents which upregulate expression of Aiolos or downregulate expression of OBF-1. Also disclosed are methods of screening using knock-out mice and B cells from knock-out mice.

Autoimmune diseases are thought to result from a breakdown in control of the immune system and its inherent tolerance to self antigens. There are several different autoimmune diseases and they affect millions of people worldwide. One or more tissues of the body is generally attacked by the immune system in autoimmune diseases. For example, in multiple sclerosis (MS), myasthenia gravis and autoimmune ureitis, the nervous system is attacked. In Crohn's disease and ulcerative colitis, the gastrointestinal system is attacked, and in psoriasis, pemphigus vulgaris and vitiligo, the skin is affected. Several autoimmune diseases attack multiple organs, for example, systemic lupus erythematosus (SLE), rheumatoid arthritis and scleroderma.

The causes of autoimmune diseases are not fully understood although there are several mechanisms thought to be involved. Normally hidden or sequestered antigens may be released into the circulation as a consequence of tissue damage or trauma. Such antigens may not be recognised as “self” antigens by the immune system because of their normal separation from T and B cells. On release into the circulation, it is thought that these antigens might elicit an immune response against normal “self” proteins.

A further possible mechanism is that antigens normally recognised as “self” become immunogenic by chemical, physical or biological alteration. Such alterations can be the result of chemicals such as drugs or even ultraviolet light, or the result of infection by a pathogenic organism which is able to modify self antigens. By being altered in any of the above ways, “self” antigens can be made immunogenic.

Cross reactivity of antibodies raised in the normal way to a foreign antigen is a further potential source of autoimmune disease, for example, antibodies against streptococcal M protein can cross react with human heart muscle.

It is also possible that a breakdown in the normal control over B or T cells is sufficient to induce an autoimmune disease. Mutation in immunocompetent cells might relieve normal checks and controls on immune cell development. It is thought that specific suppressor T cells might inhibit autoimmune reactions. Support for the idea that mutations might play a role comes from the observation that there appears to be a strong correlation of autoimmune disease and genetic relatedness. For example, the incidence of autoimmune disease is higher in identical twins than in fraternal twins. It is possible that the genetic element provides for a predisposition to the development of autoimmune disease since there also appears to be a high environmental factor in many such diseases.

Systemic lupus erythematosus (SLE) is a complex autoimmune disease and a chronic inflammatory connective tissue disorder involving multiple tissues, for example, joints, kidneys, serous surfaces and vessel walls. The disease occurs predominantly in young women and children and is characterised by the production of auto-antibodies, particularly anti-self dsDNA antibodies and antinuclear antibodies (ANA), and by immune complex-mediated glomerulonephritis (see Vyse, T. J. & Kotzin, B. L. Annu. Rev. Immunol. 16, 251-292 (1998) and Risch N. et al. J. Clin. Invest. 105, 1502-1506 (2000)). Symptoms can include cutaneous lesions including various rashes, blisters and ulcerations of the mucus membranes. Recurrent pleurisy and pericarditis are also common symptoms. Often, involvement of the central nervous system can cause other symptoms such as headaches and personality changes, stroke, epilepsy, psychosis and organic brain syndrome. Involvement of the kidneys can result in proteinuria.

Mild or remittent SLE can be treated with non-steroid anti-inflammatory drugs (NSAIDs) but high doses of these drugs in patients with SLE may cause liver toxicity. A further approach is to use anti-malarial drugs which seem to help in some patients. More severe disease is treated with corticosteroids, for example, combination of prednisolone and immunosuppressive drugs. However, corticosteroids carry likely long term complications and are preferably not used over an extended period.

Accordingly, there is a need for further active agents which can inhibit, treat, cure or prevent autoimmune diseases, for example, SLE. Preferably, such agents should be targeted at the underlying molecular basis for autoimmune disease rather than applying the general, non-specific anti-inflammatory approach of current therapies.

Wang et al (Immunity 9, 543-553, 1998) have reported the generation of homozygous knock-out mice lacking the Aiolos gene. Aiolos encodes a zinc finger DNA-binding protein that is highly expressed in mature B cells and is homologous to Ikaros. The Aiolos homozygous null-mutant mouse has B cells which exhibit an activated cell surface phenotype and undergo augmented antigen receptor (BCR)-mediated in vitro proliferation responses, even at limiting amounts of stimulant. Aiolos-deficient mice were also reported to undergo germinal centre (GC) formation and elevated serum IgG and IgE antibodies in the absence of immunisation. Auto-antibodies were also reported in aging Aiolos mutant mice as was the development of B cell Iymphomas. Additionally, B cells of the peritoneum, the marginal zone, and the recirculating bone marrow population were greatly reduced.

OBF-1 is a B-lymphocyte specific activator of octamer site mediated gene transcription which interacts with the POU domain of Oct-1 and Oct-2 in order to activate gene transcription (see WO 95/32284 derived from International Application No PCT/EP95/01834, filing dated May 15, 1995, publication dated November 1995 in the name of Ciba-Geigy).

The present inventors have investigated further the phenotype of the homozygous Aiolos mutant mice. Surprisingly, mice lacking the Aiolos transcription factor show a phenotype that corresponds very closely to SLE in humans. For example, kidney sections from Aiolos mutant mice show immunoglobulin (Ig) deposits, and heavy IgG, IgM and complement C3 deposits were identified in the glomeruli from Aiolos −/− mice. Significantly, serum auto-antibodies against dsDNA, ssDNA, histones and nuclear antigens (ANA) were also significantly higher in Aiolos −/− mice than in wild type mice. All of the mice found positive for anti-dsDNA antibody also had immune complex deposits in their kidney. Immune complex deposition in the basement membrane of the glomeruli and anti-dsDNA antibodies are important pathological features in SLE (Vyse, T J & Kotzin, B L, Annu Rev Immunol 16, 261-92 (1998) and Risch, N et al, J Clin Invest 105, 1503-1506 (2000)) and have not been previously reported in Aiolos −/− mice. In addition, the percentage of Aiolos −/− mice positive for auto-antibodies was significantly higher in female than male mice except for anti-histone antibodies. This tendency suggests that sex hormones play a role in the development of SLE, consistent with earlier observations in humans (see for example Lahita, R G, Curr Opin Rheumatol 11, 352-356 (1999)). These observations, as detailed further in the Examples below, indicate that Aiolos −/− mice represent a novel animal model for SLE.

The current inventors have crossed Aiolos homozygous knock-out mice with OBF-1 homozygous knock-out mice. Surprisingly, when Aiolos −/− mice also lack the transcriptional co-activator OBF-1, all the signs of SLE are eliminated. Moreover, at the molecular level, B cells from the double knock-out mice are no longer hyperactive, suggesting that the execution of the pathway that is triggered in an uncontrolled manner in the absence of Aiolos requires OBF-1 function. Thus, Aiolos dysfunction (mutation) in mice and other animals is expected to be involved in the pathogenesis of at least some cases of SLE. Moreover, it is also expected that the OBF-1 pathway is involved in the pathogenesis of SLE (whether Aiolos-related or not). In addition, and in view of the phenotypes observed in OBF-1 knock out mice and the double knock out mice described here, it is expected that OBF-1 and Aiolos are likely to be involved in various autoimmune diseases other than SLE, in particular, autoimmune diseases dependent upon B or T cell activation, for example, systemic autoimmune diseases such as rheumatoid arthritis or scleroderma.

Accordingly, the OBF-1 protein and gene and the Aiolos protein and gene provide new targets for agents active against autoimmune diseases, for example SLE. Suitable active agents can be identified via a number of assays of OBF-1 and Aiolos function. In particular, cell-based screening assays can be designed to determine a test agent's ability to modulate, for example, to down regulate the activity of OBF-1 (see WO 95/32284). For example, the ability of OBF-1 to activate expression of a reporter gene under control of an OBF-1 responsive element (for example the octamer motif having a consensus sequence ATGCAAAT, or its reverse complement) can be determined in the presence or absence of a test agent.

In the methods of the invention, if expression of a reporter gene linked to an OBF-1 responsive element is down-regulated in the presence of a test agent by comparison to levels in the absence of a test agent, that test agent is concluded to have a negative, or antagonistic, effect on OBF-1 mediated gene activation, and is a candidate agent active against an autoimmune disease.

Thus, in one aspect the invention provides a method of identifying an agent active against an autoimmune disease, for example, systemic lupus erythematosus (SLE), comprising providing cells containing OBF-1 protein or a fragment, variant or derivative thereof and further containing a nucleic acid comprising a nucleotide sequence encoding a reporter gene functionally linked to an OBF-1 responsive nucleotide sequence, and contacting the cells with a test agent in vitro. The level of expression of the reporter gene can be determined by comparison to a control where the cells are not contacted with the test agent.

The ability of OBF-1 to activate expression of a reporter gene can also be determined in wholly in vitro assays. Accordingly, in a further aspect, the invention provides a method of identifying an agent active against an autoimmune disease, for example SLE, comprising providing a cell extract from cells containing OBF-1 protein or a fragment, variant or derivative thereof and further containing a nucleic acid comprising a nucleotide sequence encoding a reporter gene functionally linked to an OBF-1 responsive nucleotide sequence, and subjecting the cell extract to in vitro transcription in the presence or absence of a test agent. The level of expression of the reporter gene is determined in the presence or absence of the test agent.

In one embodiment of the methods of the invention, an RNA product is determined using any suitable assay, for example reverse transcriptase polymerase chain reaction (RT-PCR), preferably quantitative or real time RT-PCR, Northern blotting, RNAse protection assays, primer extension assays or fluorescence in situ hybridisation (FISH). Other methods of specifically detecting RNA would occur to the skilled person.

In a further embodiment, a protein product of the reporter gene is determined. Protein can be detected using any suitable technique, for example electrophoresis, Western blotting, enzyme linked immunosorbent assay (ELISA) or other immunochemical methods such as immunofluorescence microscopy, light detection from e.g. luciferase or chloramphenical acetyl transferase (CAT) assays. Again, other methods will occur to the skilled person.

Various cell types from various tissues can be employed in the methods of the invention. In a preferred embodiment the cells are from a vertebrate source, for example a mammalian source. In a more preferred embodiment the cells are from a human source. In a further embodiment, the cells are from a rodent source, for example mice or rats. The cells can be primary or secondary cell strains (i.e. non-immortalised), stem cells, or established cell lines (i.e. immortalised).

In some embodiments the cells express OBF-1 naturally. Preferably, lymphoid cells are used, more preferably B cells. For example the human B lymphoid cell line Namalwa (ATCC CRL 1432) or the mouse B cell line S194 can be used. Other cell types that can be used include, but are not limited to, BJA-B human B cell lines, Molt3 and Hut78 human T cell lines, HepG2 (hepatocytes), J558L, MPC11, 70Z/3, 40E-1, 18-81 and 220-8 mouse B cell lines and spleen and peripheral blood leukocytes, thymus and small intestine cells.

In a further embodiment the cells used for the methods of the invention are engineered to express OBF-1. Such engineered cells are preferably eukaryotic (but can be prokaryotic, e.g., eubacteria for example gram positive and gram negative bacteria such as Escherichia coli strains K-12, DH5a and HB101, or Bacilli). Engineered eukaryotic cells includes yeasts such as Saccharomyces cerevisiae or Schizosaccharomyces pombe and other single celled organisms or filamentous fungi. More preferably the engineered eukaryotic cells are from higher eukaryotes, for example vertebrates, particularly mammals, and preferably from rodents such as mice or rats or from humans. Engineered cells can be transfected or transformed with OBF-1 encoding nucleic acid. In a preferred embodiment, the OBF-1 gene contained in a suitable vector or plasmid is used to transfect a suitable host cell line, for example HeLa cells or CHO cells.

DNA encoding OBF-1 may be stably incorporated into cells or may be transiently expressed using methods known in the art. Stably transfected cells may be prepared by transfecting cells with an expression vector having a selectable marker gene, and growing the transfected cells under conditions selective for cells expressing the marker gene. To prepare transient transfectants, cells are transfected with a reporter gene to monitor transfection efficiency.

Transfected and transformed cell lines expressing the OBF-1 gene can be constructed using techniques and vectors known in the art, and described in more detail in WO 95/32284 (which is hereby incorporated in its entirety). Particularly preferred vectors include expression vectors in which the OBF-1 encoding region is functionally linked to regulatory sequences such as promoter regions and enhancer regions that are capable of providing a high level of expression in the chosen host cell. An expression vector can be a recombinant DNA or RNA construct such as a plasmid, a phage, recombinant virus or other vector. It is well within the capabilities of the skilled person to use appropriate cloning and expression vectors. Vectors used to generate the cells for the methods of the invention can be constructed according to techniques very well known in the art.

The OBF-1 protein used in the methods of the invention can be full length OBF-1 protein having the sequence as set out in WO 95/32284. Accordingly, in one embodiment cells are transfected with a nucleic acid having the sequence noted in WO 95/32284, or a sequence encoding the same protein. Alternatively, fragments of the OBF-1 protein comprising the Oct-1 and/or the Oct-2 binding domains (for example, amino acids 1 to 44) can be used. Other useful fragments comprise the oct-1 and/or the oct-2 binding domains and further transcriptional activation domains from OBF-1.

In addition, variants of the OBF-1 protein can be used in the methods of the invention. Variants include proteins which exhibit substantially the same biological activity as OBF-1 protein, but which may have one or more “conservative” substitutions, deletions or additions. Less likely, a variant may have “non-conservative” changes, e.g. replacement of a glycine with a tryptophan. Guidance in determining which and how many amino acid residues may be substituted, inserted or deleted without abolishing activity may be found using computer programs well known in the art. Typically a variant will have a sequence that is at least 75% identical to the sequence given in WO 95/32284, for example 80% identical, 85% identical, 90% identical, 95% identical or 99% identical.

Variants may also be proteins encoded by a nucleic acid which hybridises under stringent conditions to the complement of the nucleic acid sequence shown in WO 95/32284. Determining appropriate stringent conditions is well within the ability of the person skilled in the art (and is discussed in, for example, Sambrook, Fritsch and Maniatis, Molecular Cloning, A Laboratory Manual). A temperature of approximately 12-20° C. below the calculated Tm of the hybrid can be used, for example, washing at approximately 68° C. in a solution containing 0.1×SSC and 0.5% SDS. Other washing buffers and conditions would occur to the skilled person.

Fusion proteins including full length OBF-1 or an active fragment or variant thereof are also envisaged as being useful in the methods of the invention. For example, tags for the targeted delivery or detection of OBF-1 can be fused to the protein, fragment or derivative. Additionally, any OBF-1 protein, fragment or variant thereof can be derivatised in any way which does not abolish its biological activity. For example, peptides having modified amino acids/peptide linkages, and peptides containing non-naturally occurring amino acids and/or cyclic peptides, which may have improved properties such as stability or activity are included.

The cells used for the methods of the invention noted above, or for production of the cell extract for the methods noted above, further require a reporter gene. The reporter gene is preferably a recombinant gene in which the OBF-1 responsive nucleotide sequence and coding sequence are heterologous to each other. Suitable reporter genes include, for example, chloramphenicol acetyltransferase (CAT), luciferase, green fluorescent protein or secreted alkaline phosphatase coding sequences functionally linked to the OBF-1 responsive element. Other reporter genes will occur to those skilled in the art. Alternatively, the reporter gene can be a gene in which the OBF-1 responsive element is normally associated with the coding region, for example, immunoglobulin genes.

In one embodiment, the OBF-1 responsive element of the reporter gene comprises an octamer motif with a consensus sequence ATGCAAAT or its reverse complement (ATTTGCAT). The reporter gene is preferably on a recombinant nucleic acid construct, for example, a DNA vector and can be a plasmid or viral based vector.

OBF-1 protein is known to activate octamer site-mediated gene transcription via its binding to the transcription factors Oct-1 and Oct-2 (see WO 95/32284), and specifically to the POU domains of those proteins. Thus, in a further aspect, the invention provides a method of identifying an agent active against an autoimmune disease, for example SLE, comprising providing OBF-1 protein or a fragment, variant or derivative thereof, oct-1 protein and oct-2 protein and a nucleic acid construct comprising a nucleotide sequence encoding a reporter gene functionally linked to an OBF-1 responsive nucleotide sequence, and subjecting the OBF-1, oct-1, oct-2 and the nucleic acid construct together to in vitro transcription in the presence or absence of a test agent. The level of expression of the reporter gene is determined in the presence or absence of the test agent.

In vitro transcription techniques are well known to those skilled in the art, and can be based for example on the method described in Dignam et al (Nucl Acids Res, 11, 1475, 1983). RNA produced by the in vitro transcription can be measured using any of the above noted methods.

Biologically active fragments of the oct-1 and/or oct-2 protein can be used in the methods of the invention. Suitable fragments include the POU domain and may further comprise any other domain to increase the transcription enhancing activity thereof (see for example Tanaker & Herr, Cell, 60, 375-386 (1990); Muller-Immergluck et al, EMBO J, 9, 1625-1634 (1990) and Tanaka et al, Mol Cell Biol, 14, 6046-6055 (1994)). In addition, biologically active variants or derivatives of the oct proteins can also be used.

The invention also provides for screens for agents capable of modulating the activity of the Aiolos protein. Thus, in accordance with a further aspect of the invention, there is provided a method of screening for an active agent capable of modifying the activity of the Aiolos protein comprising providing cells containing an Aiolos protein and a nucleic acid comprising a nucleotide sequence encoding a reporter gene functionally linked to an Aiolos responsive nucleotide sequence, contacting said cells with a test agent in vitro; and determining the level of expression of said reporter gene by comparison to a control, for example where the cells are not contacted with a test agent.

The Aiolos protein is a repressor of gene expression. Thus, if the cells contain a wild type Aiolos protein, the reporter gene is expected to be repressed under normal conditions. A test agent which causes an increase in expression of the reporter gene is concluded to be an antagonist of Aiolos. An active agent which further represses the expression of the reporter gene is concluded to be an agonist of the Aiolos protein.

Alternatively, in a further embodiment the cells can contain a mutated Aiolos protein which is normally inactive or exhibits lower repression activity than wild type Aiolos. In these cells expression from a reporter gene would be expected to be higher than in cells containing wild type Aiolos protein and this facilitates a screen for an agonist of Aiolos since an enhanced repression is easier to detect.

In a further aspect, the invention provides a method of identifying an agent active against an autoimmune disease, for example SLE, comprising providing cells containing a nucleic acid comprising a nucleotide sequence encoding a reporter gene functionally linked to an Aiolos responsive nucleotide sequence, contacting said cells with a test agent in vitro, and determining the level of expression of said reporter gene by comparison to a control where the cells are not contacted with the test agent. The cells should not contain an Aiolos protein.

In this aspect, the lack of Aiolos protein in the cells results in normal high transcription of the reporter gene. The reporter gene should be expressed under a constitutive promoter for example a viral constitutive promoter, e.g. the SV40 promoter, in any suitable cell as readily determined by a person skilled in the art. Test agents which result in a repression of the reporter gene in the absence of Aiolos protein are concluded to mimic the activity of the Aiolos protein and are therefore candidates for agents active against an autoimmune disease, for example SLE. In general, agonists of the Aiolos protein are candidate active agents active against autoimmune diseases.

In yet a further aspect, the invention provides a method of identifying an agent active against an autoimmune disease, for example, SLE comprising providing OBF-1 protein or a fragment, variant or derivative thereof and an oct protein selected from: oct-1 protein, the POU domain of the oct-1 protein, oct-2 protein or the POU domain of the oct-2 protein, and combining the OBF-1 protein with the oct protein in the presence or absence of test agent. The binding of the OBF-1 protein to the oct protein in the presence or absence of said test agent is determined.

In one embodiment the OBF-1 protein and the oct protein are combined in a cell which expresses both proteins. Extracts are prepared from the transfected cells and are assayed for binding of the OBF-1 protein to the oct protein, for example, as set out in WO 95/32284. In an alternative embodiment the OBF-1 protein and oct protein are combined in vitro. The OBF-1 proteins can be generated by recombinant means or purification using techniques known in the art.

In a further aspect, the invention provides a method of identifying an agent active against an autoimmune disease, for example SLE comprising providing cells containing OBF-1 protein or a fragment, variant or derivative thereof and an oct protein selected from: oct-1 protein, oct-2 protein, the POU domain of oct-1 protein or the POU domain of oct-2 protein. An extract (e.g. a nuclear extract) from the cells is made and is mixed with a labelled nucleic acid probe containing an oct-1 or oct-2 protein binding site, e.g. an octamer site, in the presence or absence of a test agent, and the formation of a complex between the OBF-1 protein, the oct protein and the nucleic acid probe is determined in the presence of absence of the test agent.

The formation of the complex may be assayed by subjecting the cell extract/nucleic acid probe mixture to an electrophoretic mobility shift assay. OBF-1 binding to the oct protein in the electrophoretic mobility shift assay is characterised by two shifts. One shift is caused by the oct protein binding to the oct-1 or oct-2 protein binding site on the nucleic acid probe. The second shift, of lower mobility) is due to a complex between the oct protein and the OBF-1 protein and the nucleic acid probe. A reduction in the amount of this second, so called “supershift” is indicative of a disruption of binding between the oct protein and the OBF-1 protein.

Alternatively, the formation of a complex in the methods of the invention can be determined in other ways, for example, by an enzyme linked immunoabsorbent assay, fluorescence based assays or ultra high throughput assays such as surface plasmon resonance or fluorescence correlation spectroscopy assays. Test agents which disrupt this binding capability, preferably without disrupting the binding of oct to its binding site, are expected to antagonise the biological activity of OBF-1 protein and are therefore candidates for active agents against autoimmune diseases.

In one embodiment, the cells of the assay contain both the Oct-1 protein and the Oct-2 protein (or the POU domains thereof).

Various cell types can be used in the above complex formation assays, including prokaryotic cells such as gram positive or gram negative bacteria, and eukaryotic cells such as yeasts, filamentous fungi, vertebrate primary or secondary cell strains or immortalised cell lines.

In a further preferred embodiment, agents active against autoimmune disease can be screened for by observing a down-regulation of expression of the OBF-1 gene itself. Reduced cellular OBF-1 protein levels would be predicted to result in a lower level of activation of OBF-1 activatable genes. Thus, in a further aspect, the invention provides a method of identifying an agent active against an autoimmune disease, for example, SLE comprising providing cells containing an OBF-1 gene, and contacting the cells in vitro with a test agent. The expression of the OBF-1 gene as compared to a level of expression in the absence of a test agent is determined. A test agent which causes a reduced level of expression is a candidate agent active against an autoimmune disease, for example SLE.

In addition to down-regulation of the activity or level of OBF-1, at least in some individuals, an up-regulation of expression from the Aiolos gene is also predicted to be useful in the prevention or treatment of autoimmune diseases. Accordingly, in a further aspect, the invention provides a method of identifying an agent active against an autoimmune disease, for example, SLE comprising providing cells containing an Aiolos gene, and contacting the cells in vitro with a test agent. The expression of the Aiolos gene as compared to the level of expression in the absence of a test agent is then determined. A test agent which causes an up regulation of Aiolos expression is a candidate active agent against autoimmune disease, e.g. SLE.

In one embodiment, the determination of expression of the OBF-1 gene or the Aiolos gene comprises measuring an RNA product of the OBF-1 gene or the Aiolos gene, by RT-PCR, Northern blotting, RNAse protection assays, primer extension assays or any other suitable assay as would occur to the skilled person. In a further embodiment, the determination of expression of the OBF-1 gene or the Aiolos gene comprises measuring a protein product of the OBF-1 gene or Aiolos gene, using an ELISA assay, Western blotting or light detection from e.g. luciferase or chloramphenical acetyl transferase assays or any other suitable assay as would occur to the skilled person.

The OBF-1 or Aiolos genes may be provided as heterologous nucleic acids on a vector, or may be encoded in the genome of the cells of the assay. Preferably, the expression of an endogenous OBF-1 gene or Aiolos gene is determined. Alternatively, the OBF-1 or Aiolos genes may be provided as cDNA in a vector. The vector should further contain regulatory elements from the natural gene, for example promoters, enhancers and suppressor elements (see for example Stevens et al, J Immunol, 164, 6372-6379 (2000)).

The discovery that Aiolos −/− mutant mice exhibit symptoms of SLE provides a diagnostic test for a pre-disposition to SLE (and other autoimmune diseases). Mutations in the Aiolos protein or nucleic acid sequence from an individual potentially reduce or completely nullify the activity or expression of the Aiolos protein. Any such mutant can be predicted to be diagnostic of a pre-disposition to autoimmune diseases. Accordingly, in a further aspect, the invention provides a method of diagnosing a pre-disposition to autoimmune disease, for example, SLE, comprising determining in vitro all or part of the amino acid sequence of the Aiolos protein from an individual. Thus, mutations in the DNA binding domain of Aiolos would be expected to disrupt its function and can be diagnostic of a predisposition to autoimmune disease. Similarly, mutations in binding domains to other factors, or mutations predicted to have an effect on protein stability/turnover are also predicted to be diagnostic.

In a further embodiment, the nucleic acid sequence of the Aiolos gene can be determined in order to screen for mutation in regulatory regions which might result in a reduced level of expression of the Aiolos protein.

In one embodiment, the amino acid sequence of the Aiolos protein from an individual can be deduced from a nucleic acid sequence of the individual. Alterations in the sequence over and above the wild type sequence known from Hosokawa et al, Genomics, 61, 326-329 (1999), Accession No AF129512 are indicative of a potential pre-disposition to autoimmune disease. In a further aspect, the invention also provides a method of diagnosing a pre-disposition to autoimmune disease, comprising determining the level of the expression of the Aiolos gene in a sample from an individual. In a preferred embodiment, the level of expression is measured by determining the level of messenger RNA transcribed from the Aiolos gene of the individual, more preferably the level or Aiolos protein itself can be determined in the individual.

In individuals where the expression of activity of Aiolos is reduced, or even in individuals exhibiting normal expression or activity of Aiolos, but having an autoimmune disease, for example SLE, the administration of the Aiolos protein, or active derivatives or fragments thereof may provide a route to therapy. Thus, in a further aspect, the invention provides the Aiolos protein for use in therapy and the use of the Aiolos protein in the manufacture of a medicament for treatment of an autoimmune disease, for example SLE. A nucleic acid encoding the protein for use as a pharmaceutical is also contemplated, as is the use of a nucleic acid encoding the Aiolos protein in the manufacture of a medicament for the treatment of an autoimmune disease, for example SLE.

The invention also provides pharmaceutical compositions for the prevention or treatment of autoimmune diseases, for example SLE, comprising the Aiolos protein or a fragment, variant or derivative thereof, or a nucleic acid encoding the Aiolos protein or a fragment, variant or derivative thereof.

In another aspect, the invention provides a method for the treatment of an autoimmune disease, for example SLE comprising administering an effective amount of an Aiolos protein or a fragment, variant or derivative thereof.

The determination of an effective dose is well within the capability of those skilled in the art. For any compound, the therapeutically effective dose can be estimated initially either in cell culture assays or in an appropriate animal model. The animal model is also used to achieve a desirable concentration range and route of administration. Such information can then be used to determine useful doses and routes for administration in humans.

A therapeutically effective dose refers to that amount of active agent which ameliorates the symptoms or condition. Therapeutic efficacy and toxicity of such compounds can be determined by standard pharmaceutical procedures in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage may be chosen by the individual physician in view of the patient to be treated. Dosage and administration can be adjusted to provide sufficient levels of the active moiety or to maintain the desired effect. Additional factors which may be taken into account include the severity of the disease state (e.g. tumour size and location); age, weight and gender of the patient; diet; time and frequency of administration; drug combination(s); reaction sensitivities; and tolerance/response to therapy. Long acting pharmaceutical compositions can be administered on a daily basis, every 3 to 4 days, every week, or once every two weeks, depending on half-life and clearance rate of the particular formulation.

Administration of pharmaceutical compositions of the invention may be accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, these pharmaceutical compositions can contain suitable pharmaceutically acceptable carriers comprising excipients and other compounds that facilitate processing of the active compounds into preparations which can be used pharmaceutically. Further details on techniques for formulation and administration can be found in the latest edition of Remington's Pharmaceutical Sciences (Maack Publishing Co, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, etc, suitable for ingestion by the patient.

Pharmaceutical preparations for oral use can be obtained through combination of active compounds with solid excipient, optionally grinding a resulting mixture, and processing the mixture of granules, after adding suitable additional compounds, if desired, to obtain tablets or dragee cores. Suitable excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterise the quantity of active compound (i.e. dosage).

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds can be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hanks's solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in substantial accordance with standard manufacturing procedures known in the art (e.g. by means of conventional mixing, dissolving, granulating, dragee-making, levigating, emulsifying, encapsulating, entrapping or lyophilising processes).

Aiolos −/− mutant mice are also useful for the identification of agents active against autoimmune diseases since they exhibit many symptoms of SLE and other indicators of autoimmune disease which can be readily monitored. Thus, in accordance with one aspect, the invention provides for the use of an Aiolos deficient mouse for the identification of an agent active against an autoimmune disease, for example SLE. The invention also provides for the use of Aiolos deficient B cells in vitro for the identification of an agent active against an autoimmune disease, for example SLE.

More particularly, the invention provides a method of identifying agents active against autoimmune diseases, for, example SLE comprising administering a test agent to an Aiolos deficient mouse and determining at least one effect of the test agent on symptoms of SLE in the mouse.

Several different symptoms can be monitored and the effect may be a reduction in the levels of creatine and/or urea in urine, a reduction in proteinurea in urine, a reduction in immune complex formation in the kidney, a reduction in the deposition of IgM in glomeruli of the kidney, a reduction in kidney inflammation, a reduction in spontaneous germinal centre formation, preferably in the absence of challenge of the mouse with an antigen, a reduction in serum IgG2A, IgA or IgG1, and increase in low affinity auto reactive antibodies e.g. serum IgM.

Preferably, the Aiolos deficient mouse used in such methods is female, and more preferably the methods further comprise administering the test agent to a control mouse which is not Aiolos deficient, e.g. a wild type mouse, so that the effect is determined by reference to a control.

In addition, a further aspect of the invention provides a method of identifying an agent active against an autoimmune disease, for example SLE comprising administering a test agent to an Aiolos deficient mouse, extracting B cells or B cell precursors from the mouse, culturing the B cells in vitro and determining at least one effect of the test agent on the B cell.

The invention also provides a method of identifying an agent active against an autoimmune disease, for example SLE, comprising contacting Aiolos deficient B cells with a test agent in vitro and determining at least one effect of the test agent on the B cells. Preferably the B cells are splenic or thymic B cells. The effect on B Cells (or B cell precursors) that is determined is selected from an activation of B cells, preferably determined by an increase in expression of an activation marker, a proliferation of B cells, preferably an hyperproliferation of B cells, or a reduction in antibodies reactive against dsDNA, ssDNA, histones or ANA. Other effects that can be determined are an increase in B1A cells, a reduced level of activated marker MHC II antigen or a reduction in the expression of CD23 on splenic B cells. Such effects can be determined in vitro on B cells or B cell precursors that have been contacted themselves with a test agent in vitro, or on B cells or B cell precursors derived from Aiolos deficient mice to which a test agent has been administered.

Methods of identifying an active agent against autoimmune disease, for example SLE, which involve examining the effect on B cells or B cell precursors, can further comprise control experiments comprising administering a test agent to a control mouse that is not Aiolos deficient, for example a wild type mouse. In this instance, the resulting B cell cultures are wild type and the effect of the test agent on those wild type B cells can be compared to the effect on Aiolos deficient B cells. Similarly, where the test agent is administered directly to B cells or B cell precursors, a control comprises administering the test agent to wild type B cells or B cell precursors that are not Aiolos deficient.

B cells for use in the above noted methods may be cultured in the presence of one or more mitogens, for example, anti-μ, anti-CD 40+IL4.

Preferred embodiments of the invention will now be described by way of example and with reference to the drawings:—

FIG. 1 shows immune complex-mediated glomerulonephritis in Aiolos deficient mice. (a) shows immunofluorescence staining of kidney cryosections with FITC-conjugated anti-mouse IgG, IgM or C3 antibodies deposits of IgG, IgM and C3 are present in glomeruli of Aiolos −/− mice, but not in aged-matched wild-type mice or double mutant mice. (b) shows PAS staining of paraffin-embedded sections. Wild-type mouse shows a normal glomerulus. Sections from a Aiolos −/− mice show sclerotic and enlarged glomeruli characteristic of severe inflammation. No inflammation was visible in double deficient mice.

FIG. 2 shows that Aiolos −/− mice develop renal failure. Serum levels of Creatinine (a) and Urea (b) in 24 Aiolos −/− mice aged 3 to 5 month old and 11 age-matched control mice show that the values of Creatinine and Urea from 7 Aiolos −/− mice are over the mean plus 2S.D. of the wild-type group. 5 out of these 7 mice had elevated levels of both markers simultaneously.

FIG. 3 shows B cell development in mice of the different genotypes. (a) Bone marrow cells double-stained with the indicated antibodies are analyzed by flow cytometry. (b) Splenic cells analyzed by single-color flow cytometry. The percentage of B220+ cells is indicated. Genotypes are indicated above the panels. Data are representative of six mice for each genotype, aged 6-10 weeks.

FIG. 4 shows B cell activation, proliferation and immunoglobulin production. (a) B cell activation assayed by flow cytometry for expression of the activation marker CD23. Splenocytes were stained with B220-FITC and CD23-PE. Only B220+ cells were gated and 2×10⁴ cells were collected. B cells expressing high levels of CD23 are framed and the corresponding percentage is given. Results are representative of three animals for each genotype. (b) B cell proliferation in vitro determined by measuring incorporation of ³H-thymidine. Purified splenic B cells were cultured under the conditions indicated. For each stimulus the value (cpm) obtained with wild-type cells was set to 100% and the values obtained with mutant cells are given relative to this. The means±S.D. from 3 mice assayed in triplicate is shown in (c) and (d). Serum immunoglobulins quantitated by ELISA. In each case, sera from six mice 2 to 4 months old were assayed.

FIG. 5 shows that germinal center formation requires OBF-1 even in Aiolos mutant mice. Splenic B cell follicles and GCs were stained with anti-mouse IgM-rodamin (red) and PNA-biotin, respectively. The latter was developed by streptavidin-FITC (green). A representative section from each genotype is shown.

Materials and Methods

Mice

Aiolos deficient mice (Wang, J H et al. Immunity 9, 543-553 (1998)) were crossed with OBF-1 −/− mice (Schubart, D B, et al. Nature 383, 538-542 (1996)) in order to generate double heterozygotes. Interbreeding these heterozygotes was used to generate double knockout mice as well as the corresponding controls. The genotype of the mice was determined by PCR of tail DNA. All the mice were maintained in a conventional facility.

Immunohistochemistry and Histopathology

Kidney and spleen were embedded in OCT compound (Miles). For detection of immune complexes, frozen sections were stained with anti-IgG-FITC, anti-IgM-FITC (Southern Biotechnology) and anti-complement 3-FITC (Cappel). For spleen staining, B cell follicles and GCs were revealed by anti-IgM-Rodamin (Southern Biotechnology) and biotinylated peanut agglutinin (PNA) (Vector), respectively. The latter was developed by streptavidin-FITC (Southern Biotechnology). For histological examination, kidneys were embedded in paraffin and sections were stained with Periodic Acid Schiff (PAS).

Autoantibodies and Ig Detection by ELISA

Auto-antibodies were detected by ELISA using plates pre-coated for ssDNA, histone, ANA (Euroimmune) and dsDNA (Euroimmune, Sigma). Serum dilution used was 1:50. For some double mutant mice, 1:5 dilution of serum has been used. Anti-mouse IgG labeled with horseradish peroxidase (HRP, Santa Cruz) was used as secondary antibody. The mean plus two standard deviations of the OD obtained with wild-type serum was set as the lower limit for a sample to be scored positive. Immunoglobulins were quantitated by ELISA using capture antibodies anti-IgM, anti-IgA, anti-IgE, anti-IgG and anti-IgG subclasses (Southern Biotechnology). Relative isotypes antibodies conjugated with AP were used as second antibody.

Flow Cytometric Analysis

Single cell suspension was prepared from spleen and bone marrow. The following anti-mouse antibodies were used to detect surface markers in direct or indirect immunofluorescence: anti-CD45R-FITC (B220), anti-c-kit-biotin, anti-CD25-biotin (TAC), anti-IgM-biotin, anti-IgD-biotin, anti-CD23-PE and anti-CD40L-PE. Biotinylated antibodies were developed with streptavidin-PE. 3×10⁵ events were collected on the lymphocyte gate using a FACScalibur.

B Cell Isolation and Proliferation Assay

Splenic B cells were purified by negative selection using a B cell isolation kit (Cytovax Biotechnologies Inc.). 5×10⁵ ml⁻¹ B cells were cultured in triplicate with the indicated stimuli for 72 h, pulsed with 1 μCi of methyl-³H thymidine for the last 12 h. Incorporation of isotope was measured by scintillation counting.

### EXAMPLE 1

### Aiolos(−/−)Mice Have Immune Complexes in the Kidney and are Positive for Serum Autoantibodies

Aiolos mutant mice (−/−) were generated according to Wang, J H et al. Immunity 9, 543-553 (1998). Kidney sections from these Aiolos mutant mice were stained for the presence of immune complex. As shown in FIG. 1a, heavy IgG, IgM and complement (C) 3 deposits were identified in the glomeruli from Aiolos −/−, but not from WT mice. Serum autoantibodies were also detected and the percentage of Aiolos mutant mice positive for autoantibodies against dsDNA, ssDNA, histones and ANA was significantly higher than in age-matched wild-type mice: 82% of Aiolos mutant mice produced anti-dsDNA antibody (Table 1). All mice positive for anti-dsDNA antibody also had immune complex deposits in their kidney. Furthermore, the percentage of Aiolos −/− mice positive for autoantibodies was significantly higher in female than in male, except for anti-histones (Table 2). This tendency is consistent with earlier observations (Lahita, R. G. Curr Opin Rheumatol 11, 352-356 (1999)), suggesting that sex hormones play a role in the development of SLE. In addition, kidney histological staining demonstrated severe inflammation caused by immune complex deposits in the form of segmental or enlarged glomeruli (FIG. 1b).

TABLE 1
Autoantibodies production in Aiolos-deficient and wild-type mice

| | |
|---|---|
| Males | Females |

| | Wt | Aiolos −/− | Wt | Aiolos −/− |
|---|---|---|---|---|
| Anti-dsDNA | 6(16) | 77(26) | 13(15) | 87(23) |
| Anti-ssDNA | 30(10) | 56(16) | 27(11) | 70(10) |
| Anti-ANA | 30(10) | 53(17) | 18(11) | 89(9) |
| Anti-histone | 10(10) | 53(15) | 27(11) | 33(9) |

Percentages of positive mice for autoantibodies are shown. The number of used mice ranging from 3 to 7 months is given in parentheses. Serum autoantibodies were detected by ELISA. The mean of wild-type mice plus two standard deviations was set as the lower limit for positive samples.

TABLE 2
Failure to produce autoantibodies in Aiolos −/− OBF-1 −/− mice

| | Aiolos −/− | Aiolos −/− OBF-1 −/− |
|---|---|---|
| Anti-dsDNA | 82(49) | 0(31) |
| Anti-ssDNA | 62(26) | 0(28) |
| Anti-ANA | 65(26) | 0(28) |
| Anti-histone | 49(24) | 0(28) |

Percentages of positive mice for autoantibodies are shown. The number of used mice ranging from 3 to 7 months is given in parentheses. Serum autoantibodies were detected by ELISA. The mean of wild-type mice plus two standard deviations was set as the lower limit for positive samples.

### EXAMPLE 2

### Aiolos(−/−) Mice Have Significantly Increased Levels of Creatinine and/or Urea in their Serum

Severe glomerulonephritis can lead to chronic renal failure. Accordingly, the serum levels of Creatinine and Urea, which are indicators for renal failure, were measured. Seven out of 24 Aiolos −/− mice 3 to 5 months old had significantly increased levels of Creatinine (FIG. 2a) and/or Urea (FIG. 2b) compared to the age-matched control group. Moreover, 5 out of these 7 mice had elevated levels of both markers simultaneously. Proteinurea has also been detected in several of these mice.

### EXAMPLE 3

### Aiolos −/−, OBF-1 −/− Double Mutant Mice Lack Autoantibodies and Show No Immune Complex Deposits in the Glomeruli

Aiolos −/− mice were crossed with OBF-1 −/− mice and autoantibodies against dsDNA, ssDNA, histone and ANA were detected in the serum of double mutant mice. As shown in Table 2, the double knockout mice completely lacked autoantibodies, even at the lowest serum dilution (1:5) tested. This result indicates that OBF-1 is essential for development of the autoimmune responses observed in Aiolos −/− mice. Furthermore, In agreement with the above observation, no immune complex deposits were found in the glomeruli of Aiolos −/− OBF-1 −/− mice (FIG. 1a) and histological examination of kidney morphology showed normal glomeruli in these mice (FIG. 1b). All these results support the conclusion that lack of OBF-1 prevents the development of SLE in Aiolos −/− mice.

### EXAMPLE 4

### B-Cell Development is Severely Impaired at the Immature Stage in the Double Knockout Mouse

The numbers of pro-B (B220+c-kit+) and pre-B (B220+Tac+) cells in bone marrow appeared normal in all mutant mice. However, B cell development was severely impaired at the immature stage (B220+IgM+) in the double knockout mice (FIG. 3a), but not in the single mutants in agreement with earlier results (Wang, J. H. et al. Immunity 9, 543-553 (1998) and Schubart, D. B., et al. Nature 383, 538-542 (1996)). This affected heavily further maturation to IgD+ B cells which were largely missing (FIG. 3a). Furthermore, CD23+B cells, which represent mature B cells in bone marrow, were also drastically reduced in the double mutant mice (FIG. 3a). The strongly reduced number of B220+IgM+ cells in bone marrow is not the result of impaired μ chain gene transcription because cytoplasmic μ chain expression was not affected in the double mutant mice. In addition, the number of splenic B220+ cells was found to be reduced about 2 fold in the double mutant mice (FIG. 3b). Our data indicate that efficient transition from the pre-B to the immature B cell stage requires either Aiolos or OBF-1.

### EXAMPLE 5

### CD23 Expression is Elevated on Splenic B-Cells in Aiolos −/− Mice But not in Double Knockout Mutant Mice

To further define the role of B cell in the development of SLE, the expression of the activation marker CD23 was analysed. FIG. 4a shows that CD23 expression on splenic B cells in Aiolos −/− mice was elevated compared to WT mice. This is in accordance with earlier result that Aiolos −/− B cells expressed high level of activated marker MHC II antigen (Wang, J. H. et al. Immunity 9, 543-553 (1998)). By contrast, in OBF-1 deficient mice CD23 expression was reduced. When both proteins were missing simultaneously, CD23 expression became close to the level of wild-type, indicating that lack of OBF-1 antagonizes the defect caused by the absence of Aiolos and inhibits B cell activation in Aiolos −/− mice. Interestingly, up-regulation of CD23, which is a low-affinity receptor (FcμRII) for IgE and regarded to be a negative regulator of IgE production (Stief, A. et al. J Immunol 152, 3378-3390 (1994) and Riffo-Vasquez, Y. et al. Clin Exp Allergy 30, 728-738 (2000)], did not prevent elevated production of IgE in Aiolos mutant mice (FIG. 4d).

### EXAMPLE 6

### Hyperproliferation of B-Cells Exhibited by Aiolos −/− Mice is Weakened in the Double Knockout B-Cells

The ability of purified splenic B cells of the different genotypes to proliferate in vitro in response to mitogens such as LPS, anti-μ or anti-CD40+IL4 was examined. In all cases except LPS stimulation, Aiolos −/− B cells showed an increased ³H-thymidine uptake compared to WT cells (FIG. 4b), in good agreement with earlier findings (Wang, J. H. et al. Immunity 9, 543-553 (1998)). Aiolos-deficient B cells can be driven to proliferate by anti-μ even at concentrations that do not activate wild-type cells, suggesting that lack of Aiolos lowers the threshold of the BCR pathway (Wang, J. H. et al. Immunity 9, 543-553 (1998)). This hyperproliferation is weakened in the double knockout B cells (FIG. 4b), indicating that normal OBF-1 function is necessary for the hyperactivity of Aiolos-deficient B cells. In addition, splenic B cells in OBF-1 −/− mice have a poor response to anti-μ stimulation (FIG. 4b); thus, failure of BCR-mediated signalling could contribute to the impaired TI and TD responses in OBF-1 −/− mice.

### EXAMPLE 7

### Elevated Serum Levels of Immunoglobulins in Aiolos −/− Mice are Decreased in the Double Knockout Mouse

Abnormal autoantibody production in the Aiolos and double mutant mice prompted us to examine serum Ig levels. Although Aiolos-deficient mice do not have increased total IgG levels, they show elevated serum IgG2a, IgA and IgG1 and also reduced serum IgM (FIG. 4c) (Wang, J. H. et al. Immunity 9, 543-553 (1998)). However, in the double knockout mice the levels of all the Igs decreased close to those found in the OBF-1 deficient mice. B1a cells are one of the main sources for low-affinity autoreactive IgM antibodies (Murakami, M., Honjo, T. Immunol Today 16, 534-539 (1995)). Aiolos −/− mice have greatly reduced B1a cells, and this may account for the reduced serum IgM (Wang, J. H. et al. Immunity 9, 543-553 (1998)). Interestingly, Aiolos −/− mice still have IgM deposition in the glomeruli in spite of reduced serum IgM.

### EXAMPLE 8

### Mice Lacking Aiolos and OBF-1 Fail to Form Germinal Centres

Mice deficient for Aiolos form GCs in absence of antigen challenge (FIG. 5, Wang, J. H. et al. Immunity 9, 543-553 (1998)). This persistent GC formation may contribute to the development of SLE in these mice. Multiple evidences demonstrated that CD40L expression on activated T cells is crucial to trigger B cells to form GC (Xu, J. et al. Immunity 1, 423-431 (1994) and Grammer, A. C., et al. J Immunol 163, 4150-4159. (1999)). High expression of CD40L on activated as well as unstimulated T and B cells from lupus patients has been reported (Desai-Mehta, A., et al. J. Clin. Invest. 97, 2063-2073 (1996) and Koshy, M., et al. J Clin Invest. 98, 826-837 (1996)). To understand the mechanism of spontaneous GC formation in Aiolos −/− mice, CD40L expression was detected. No CD40L expression was observed on unstimulated T cells (from thymus and spleen) and on splenic B cells from Aiolos-deficient and wild-type mice. However, after activation by PMA plus ionomycin, Aiolos −/− T cells express CD40L similar to WT cells. These results indicate that dysregulation of CD40L expression is not responsible for the spontaneous and persistent GC formation in Aiolos −/− mice and suggest that other molecule(s) are implicated in the phenotype. Previous results showed that OBF-1 is essential for GC formation (Schubart, D. B., et al. Nature 383, 538-542 (1996)). Mice lacking both Aiolos and OBF-1 completely fail to form GC (FIG. 5). This suggests that OBF-1 and/or its target gene(s) lie downstream of Aiolos and are required for GC formation in Aiolos deficient mice.

1. A method of identifying an agent active against an autoimmune disease, for example, systemic lupus erythematosus (SLE) comprising:—
a) providing cells containing:—
(i) OBF-1 protein or a fragment, variant or derivative thereof;
(ii) a nucleic acid comprising a nucleotide sequence encoding a reporter gene functionally linked to an OBF-1 responsive nucleotide sequence;
b) contacting said cells with a test agent in vitro; and
c) determining the level of expression of said reporter gene by comparison to a control where the cells are not contacted with the test agent.

2. A method of identifying an agent active against an autoimmune disease, for example, SLE comprising:—
a) providing a cell extract from cells containing:
(i) OBF-1 protein or a fragment, variant or derivative thereof;
(ii) a nucleic acid comprising a nucleotide sequence encoding a reporter gene functionally linked to an OBF-1 responsive nucleotide sequence;
b) subjecting said cell extract to in vitro transcription in the presence or absence of a test agent; and
c) determining the level of expression of said reporter gene in the presence or absence of said test agent.

3. A method of identifying an agent active against an autoimmune disease, for example SLE, comprising:—
a) providing OBF-1 protein or a fragment, variant or derivative thereof, oct-1 protein and oct-2 protein and a nucleic acid comprising a nucleotide sequence encoding a reporter gene functionally linked to an OBF-1 responsive nucleotide sequence; and
b) subjecting said OBF-1, oct-1, oct-2 and said nucleic acid construct together to in vitro transcription in the presence or absence of a test agent; and
c) determining the level of expression of said reporter gene in the presence or absence of said test agent.

4. A method of screening for an agent capable of modulating the activity of the Aiolos protein comprising:—
a) providing cells containing:—
(i) an Aiolos protein;
(ii) a nucleic acid comprising a nucleotide sequence encoding a reporter gene functionally linked to an Aiolos responsive nucleotide sequence;
b) contacting said cells with a test agent in vitro; and
c) determining the level of expression of said reporter gene by comparison to a control where the cells are not contacted with a test agent.

5. A method as claimed in claim 4 wherein the Aiolos protein is a mutated, inactive or partially inactive form of Aiolos protein.

6. A method of identifying an agent active against an autoimmune, for example SLE, comprising:—
a) providing cells containing a nucleic acid comprising a nucleotide sequence encoding a reporter gene functionally linked to an Aiolos responsive nucleotide sequence;
b) contacting said cells with a test agent in vitro; and
c) determining the level of expression of said reporter gene by comparison to a control where the cells are not contacted with the test agent;
wherein said cells do not contain an Aiolos protein.

7. A method as claimed in any of claims 1 to 6 wherein said determining step comprises measuring an RNA product of said reporter gene, for example by reverse transcriptase polymerase chain reaction (RT-PCR), quantitative RT-PCR, real time RT-PCR, Northern blotting, RNAse protection assays, primer extension assays or fluorescence in situ hybridisation (FISH).

8. A method as claimed in claim 1 or any of claims 4 to 6 wherein said determining step comprises measuring a protein product of said reporter gene.

9. A method as claimed in any of claims 1 to 3, 7 or 8 wherein said OBF-1 responsive element comprises an octamer motif with the consensus sequence ATGCAAAT or its reverse complement (ATTTGCAT).

10. A method as claimed in any of claims 1 to 9 wherein said reporter gene is contained in a vector for example a plasmid or viral vector.

11. A method as claimed in any of claims 1 to 9 wherein said reporter gene is selected from Chloramphenicol acetyl transferase (CAT), luciferase, green fluorescent protein (GFP) or secreted alkaline phosphatase (SEAP).

12. A method as claimed in any of claims 1 to 11 wherein said cells are eukaryotic, and are selected from yeasts, filamentous fungi, primary or secondary cell strains or immortalised cell lines.

13. A method as claimed in any of claims 1 to 12 wherein said cells are of human origin.

14. A method as claimed in any of claims 1 to 12 wherein said cells are of rodent origin, for example, from rats or mice.

15. A method of identifying an agent active against an autoimmune disease, for example, SLE comprising:—
a) providing OBF-1 protein, or a fragment, variant or derivative thereof;
b) providing an oct protein selected from: oct-1 protein, the POU domain of the oct-1 protein, oct-2 protein or the POU domain of the oct-2 protein;
c) combining said OBF-1 protein with said oct protein in the presence or absence of test agent; and
d) determining binding of said OBF-1 protein to said oct protein in the presence or absence of said test agent.

16. A method as claimed in claim 15 wherein said binding is determined by any one or more of an enzyme linked immunoabsorption assay (ELISA), electrophoretic mobility shift assay (EMSA), fluorescence based assays such as fluorescence resonance energy transfer (FRET), surface plasmon resonance (SPR) or fluorescence correlation spectroscopy (FCS).

17. A method as claimed in claim 15 or claim 16 wherein said OBF-1 protein and said oct protein are combined in vitro.

18. A method of identifying an agent active against an autoimmune disease, for example SLE comprising:—
a) providing cells containing:—
(i) OBF-1 protein, or a fragment, variant or derivative thereof;
(ii) an oct protein selected from: oct-1 protein, oct-2 protein, the POU domain of oct-1 protein or the POU domain of oct-2 protein;
b) preparing an extract from said cells;
c) mixing said extract with a labelled nucleic acid probe containing an oct-1 or oct-2 protein binding site, e.g. an octomer site, in the presence or absence of a test agent; and
d) determining the formation of a complex between said OBF-1 protein, said oct protein and said nucleic acid probe in the presence or absence of the test agent.

19. A method as claimed in claim 18 wherein said determining step d) comprises subjecting said extract/nucleic acid probe mixture to an electrophoretic mobility shift assay.

20. A method as claimed in claim 18 wherein said determining step comprises an assay selected from an enzyme linked immunoabsorption assay (ELISA), fluorescence based assays and ultra high throughput assays, for example surface plasmon resonance (SPR) or fluorescence correlation spectroscopy (FCS) assays.

21. A method as claimed in any of claims 18 to 20 wherein oct-1 (or the POU domain of oct-1) and oct-2 (or the POU domain of oct-2) are contained in said cells.

22. A method as claimed in any of claims 18 to 21 wherein said extract is a nuclear extract.

23. A method as claimed in any of claims 18 to 21 wherein said cells are prokaryotic, for example gram positive or gram negative bacteria.

24. A method as claimed in any of claims 18 to 22 wherein said cells are eukaryotic for example, yeast, filamentous fungi, primary or secondary cell strains or immortalised cell lines.

25. A method of identifying an agent active against an autoimmune disease, for example, SLE comprising:—
a) providing cells containing an OBF-1 gene
b) contacting said cells in vitro with a test agent; and
c) determining expression of said OBF-1 gene as compared to a level of expression in the absence of a test agent.

26. A method of identifying an agent active against an autoimmune disease, for example, SLE comprising:—
a) providing cells containing an Aiolos gene;
b) contacting said cells in vitro with a test agent; and
c) determining the expression of said Aiolos gene as compared to the level of expression in the absence of a test agent.

27. A method as claimed in claim 25 or claim 26 wherein said determining step c) comprises measuring an RNA product.

28. A method as claimed in claim 25 or claim 26 wherein said determining step c) comprises measuring a protein product.

29. A method as claimed in claim 25 wherein said OBF-1 gene is provided in on a vector.

30. A method as claimed in claim 25 wherein said OBF-1 gene is encoded in the genome of said cells

31. A method as claimed in claim 26 wherein said Aiolos gene is provided on a vector.

32. A method as claimed in claim 26 wherein said Aiolos gene is encoded by the genome of said cells.

33. A method of diagnosing a pre-disposition to autoimmune disease, for example, SLE, comprising determining in vitro all or part of the amino acid sequence of the Aiolos protein from an individual.

34. A method as claimed in claim 33 wherein the amino acid sequence is deduced from a nucleic acid sequence of said individual.

35. A method of diagnosing a pre-disposition to autoimmune disease, comprising determining the level of the expression of the Aiolos gene in a sample from an individual.

36. A method as claimed in claim 35 wherein said determining step comprises determining the level of a nucleic acid.

37. A method as claimed in claim 35 or claim 36 wherein said determining step comprises determining the level of the Aiolos protein.

38. The Aiolos protein or a derivative or fragment thereof for use as a pharmaceutical.

39. A nucleic acid encoding the Aiolos protein for use in therapy.

40. The use of the Aiolos protein in the manufacture of a medicament for the treatment of an autoimmune disease, for example, SLE.

41. The use of a nucleic acid encoding the Aiolos protein in the manufacture of a medicament for treatment of an autoimmune disease, for example, SLE.

42. A pharmaceutical compositions for the prevention or treatment of autoimmune diseases, for example SLE, comprising the Aiolos protein or a fragment, variant or derivative thereof.

43. A pharmaceutical compositions for the prevention or treatment of autoimmune diseases, for example SLE, comprising a nucleic acid encoding the Aiolos protein or a fragment, variant or derivative thereof.

44. A method for the treatment of an autoimmune disease, for example SLE comprising administering an effective amount of an Aiolos protein or a fragment, variant or derivative thereof.

45. A method for the treatment of an autoimmune disease, for example SLE comprising administering an effective amount of a nucleic acid encoding the Aiolos protein or a fragment, variant or derivative thereof.

46. A method of identifying an agent active against an autoimmune disease, for example SLE, comprising administering a test agent to an Aiolos deficient mouse and determining at least one effect of the test agent on symptoms of SLE in the mouse.

47. A method as claimed in claim 46, further comprising administering the test agent to a control mouse which is not Aiolos deficient so that the effect is determined by reference to the control, optionally wherein the control mouse is a wild type mouse.

48. A method as claimed in claim 46 or claim 47, wherein the Aiolos deficient mouse is female.

49. A method as claimed in any of claims 46 to 48, wherein the effect is any one or more of:
a reduction in levels of creatinine and/or urea in urine,
a reduction in proteinurea in urine,
a reduction in immune complex formation in the kidney,
a reduction in the deposition of IgM in glomeruli of the kidney,
a reduction in kidney inflammation,
a reduction in spontaneous germinal centre formation, preferably in the absence of challenge of the mouse with an antigen,
a reduction in serum IgG2A, IgA or IgG1,
an increase in low affinity autoreactive antibodies, e.g. serum IgM.

50. A method of identifying an agent active against an autoimmune disease, for example SLE, comprising administering a test agent to an Aiolos deficient mouse, extracting B cells or B cell precursors from the mouse, culturing the B cells in vitro and determining at least one effect of the test agent on the B cells.

51. A method as claimed in claim 50, further comprising administering a test agent to a control mouse that is not Aiolos deficient, extracting control B cells or B cell precursors from the mouse, culturing the B cells in vitro and determining at least one effect of the test agent on the B cells, optionally wherein the mouse and the resultant B cell cultures are wild type.

52. A method as claimed in claim 46 or claim 51, wherein the mouse is female.

53. A method as claimed in any of claims 46 to 52, wherein the test agent is administered intravenously.

54. A method of identifying an agent active against an autoimmune disease, for example SLE, comprising contacting Aiolos deficient B cells with a test agent in vitro and determining at least one effect of the test agent on the B cells.

55. A method as claimed in claim 54 further comprising contacting control B cells that are not Aiolos deficient with the test agent in vitro so that the effect is determined by reference to the control, optionally wherein the control B cells are wild type.

56. A method as claimed in claim 54 or claim 55, wherein the Aiolos deficient B cells have a female genotype.

57. A method as claimed in any of claims 50 to 56, wherein the B cells are splenic or thymic B cells.

58. A method as claimed in any of claims 46 to 57, wherein the effect is any one or more of:
an activation of B cells, preferably determined by an increase in expression of an activation marker,
a proliferation of B cells, preferably an hyperproliferation of B cells,
a reduction in antibodies reactive against dsDNA, ssDNA histones or ANA,
an increase in B1a cells,
a reduced level of activated marker MHC II antigen, or
a reduced expression of CD23 on splenic B cells.

59. A method as claimed in any of claims 50 to 58, wherein the B cells are cultured in the presence of one or more mitogens.

60. A method as claimed in claim 59, wherein the mitogens are selected from anti-μ, anti-CD40+ IL4.

61. The use of an Aiolos deficient mouse for the identification of an agent active against an autoimmune disease, for example SLE.

62. A use as claimed in claim 61, wherein the identification of the agent active against SLE is performed according to a method of any of claims 46 to 53 or 58 to 60.

63. The use of Aiolos deficient B cells in vitro for the identification of an agent active against an autoimmune disease, for example SLE.

64. A use as claimed in claim 63, wherein the identification of the agent active against SLE is performed according to a method of any of claims 54 to 60.
